(19) United States
(12) Patent Application Publication
(10) Pub. No.: US 2005/0214381 A1
(43) Pub. Date: Sep. 29, 2005

(54) Method for improvement and modulation of humane immune response through supplementation of colostrum

(76) Inventor: Richard Cockrum, Perry, IA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/087,364

(22) Filed: Mar. 23, 2005

Related U.S. Application Data
(62) Division of application No. 10/340,953, filed on Jan. 13, 2003, now abandoned.

(51) Int. Cl. A61K 35/20

(52) U.S. Cl. 424/535

(57) ABSTRACT

The present invention establishes a dose dependant correlation between the amount of bovine colostrum consumed as a supplement and the levels of increase of immunoglobulins and lymphocytes present in the immune system of the studied subjects. In this way the invention develops a method of enhancing the immune response in an individual by supplementation of bovine colostrum.

### BACKGROUND OF THE INVENTION

1. Field of the Invention

The invention relates generally to a method for enhancing the immune response of humans using colostrum and, more specifically, to a method for increasing the immune response using an increased dosage of bovine colostrum.

2. Background

Colostrum, the first milk produced after birth, is a thick yellow fluid produced from the mammary glands during the first hours after birth and acts as the specific diet of mammalians neonates (Zaremba et al., 1993; Yvon et al., 1993; Wells et al., 1996; Morin et al., 1997; and Fetherston et al., 2001). It is a source of nutrients such as proteins, carbohydrates, fat, vitamins, and minerals, and also contains several biologically-active molecules which are essential to the body's immune and growth functions. It is an ideal substance that offers anti-aging hormones in perfect combination and works naturally to replace both the body's own growth factors and stimulate the endocrine system to continue producing these vital and revitalizing hormones (Rudman et al., 1981; Cappon et al., 1994; and Blum & Baumrucker 2002).

Growth factors in an individual's body stimulate normal growth, as well as help regenerate and accelerate the repair of aged or injured muscle, skin collagen, bone, cartilage and nerve tissues, and also stimulate the body to burn fat for fuel instead of the body's own muscle tissue, build lean muscle, and can be used as an effective topical application for burns, injuries and skin rejuvenation. There is evidence that growth factors and immune boosting agents present naturally in colostrum work synergistically to form a potent anti-aging and endocrine modulation factor (Davidson, 1989; Wilson, 1998; and Solomons, 2002). Due to its high content of Insulin-like Growth Factors (IGF), colostrum has been shown to help maintain blood glucose levels to serve the brain. Also, Insulin-like Growth Factors from colostrum has been shown to cross the blood brain barrier to help nerve synapse in the brain and therefore enhancing mental acuity (Collier et al., 1991; Donovan & Odle, 1994; Koletzo et al., 1998; and Kuipers et al., 2002). Colostrum also stimulates the brain to release serotonin and dopamine and prolong their uptake in addition to activate the release of other chemicals from the brain that affect alertness and concentration (Korhonen et al., 2001; and Henderson, 2002). Colostrum is also a unique agent for supporting the body's basic functions due to its high contents of growth factors that are involved in both DNA and RNA synthesis and promoting normal cell growth and tissue repair (Kiecolt-Glaser & Glaser, 1991; Kishikawa et al., 1996; and Kuhne et al., 2000). Colostrum supports human organs by providing both multiple powerful immune factors (natural antibodies) and many of growth factors that work together to restore potent functions which improve the physical performance of humans (Francis, 1986; Koldovsky et al., 1991; Nitsch & Nitsch, 1998; Kelleher & Lonnerdal, 2001; and Coombes et al., 2002).

Similar to human colostrum, bovine colostrum is the first milk secreted in the first few days after calving (Campbell & Petersen 1963; Brambell 1969; and Butler 1974). It has been historically conceptualized that bovine colostrum could be served as a healthy human food or supplement due to its nutritional benefits in bovine animals (Lascelles, 1963; Lamm et al., 1978; Butler, 1986; Larson, 1992; and Quigley & Drewry, 1998). Growth factors found in bovine colostrum have been found to be virtually identical to those in human colostrum, and the immune factors in bovine colostrum are reportedly four times richer than the human colostrum. Therefore, bovine colostrum has been reported to provide stimulation of visceral organs and skeletal muscle protein synthesis (Burns et al., 1992), improvement of general physical performance and running activities (Buckley et al., 1998, 1999, 2000, and Antonio et al., 2000), and pronounced immune defenses against many of infectious diseases due to its high contents of immunoglobulins and modulation of humoral immune responses (Hanson et al., 1985; Antonius et al., 2000; Craczyk et al., 2000; and He et al., 2001). Bovine colostrum also contains many bioactive constituents that make it useful for human applications (Pakkanen & Aalto, 1997; Schlimme et al., 2000; and Korhonen et al.; 2001).

It has long been recognized that breast feeding colostrum in humans could offer a pronounced enhancement of passive immunity for humans primarily via transfer of lacteal specific and non-specific antibodies directly to infants (Reddy et al., 1988; Davidson, 1996; and Korhonen, 1998). Furthermore, regular consumption of bovine colostrum has been reported as a protective role for infantile gastrointestinal immunity due to its direct effect on promoting the development of infantile gut-associated lymphoid tissues, which are responsible mainly for providing infantile gut immunity (Carbonare et al., 1997; Kelly & Coutts, 2000; and Korhonen et al., 2000).

Bovine colostrum contains a level of immunoglobulins that is several hundred times higher than ordinary bovine milk (Butler, 1994; and Korhonen et al., 1995). It also contains a series of physiologically bioactive growth promoting factors that act as mediators of infantile growth and development (Reiter, 1978; Donovan & Odle, 1994; and Merro et al., 1997) and a series of anti-microbial fractions including lactoferrin, prostaglandin, lactoperoxidase, and lysozymes (Sanchez et al., 1992; Levay & Viljion, 1995; Lonnerdol & Lyer, 1995; and Antonuis et al.; 2000). In this way, studies postulate that there are possible therapeutic benefits of orally ingested bovine colostrum in infants in protecting against bacterial and viral gastrointestinal infectious diseases until their own immunity system develops and matures (Nord et al., 1990; Mitra et al, 1995; and Warmey et al., 1999).

Because of the recognized benefits of colostrum there is a desire to establish a method of providing an increased immunity for individuals that have a low immunity, such as infants or children and people with an immune response disease. Although some benefits of bovine colostrum have been recognized in bovine animals, the application as a supplement in humans as well as the recommended dose to be administered for providing the maximum enhanced human immune response have not been made apparent.

### SUMMARY OF THE INVENTION

The object of this invention is to establish a dose dependant correlation between the amount of bovine colostrum consumed and the levels of immunoglobulins and lymphocytes present in the human immune system.

These and other objects of the present invention will become apparent to those skilled in the art upon reference to the following specification, drawings, and claims. To that end, this invention pertains to a method for enhancing the human immune response administering at least 300 mg per day of bovine colostrum to a human subject. This method of enhancing the human immune response through the supplementation of bovine colostrum increases the level of immunoglobulins and the level of lymphocytes present in an individual's blood serum.

### BRIEF DESCRIPTION OF THE DRAWINGS

FIG. 1 shows the average level of IgG present in blood samples taken from subjects in a study of the effects of bovine colostrum supplementation.

FIG. 2 shows the average level of IgA present in blood samples taken from subjects in the study of the effects of bovine colostrum supplementation.

FIG. 3 shows the average level of IgM present in blood samples taken from subjects in the study of the effects of bovine colostrum supplementation

FIG. 4 shows the average level of CD4 present in blood samples taken from subjects in the study of the effects of bovine colostrum supplementation.

FIG. 5 shows the average level of CD8 present in blood samples taken from subjects in the study of the effects of bovine colostrum supplementation.

FIG. 6 shows the combined results of immunoglobulin and lymphocyte levels present in blood samples taken from subjects in the study of the effects of bovine colostrum supplementation.

FIG. 7 shows the combined results of immunoglobulin and lymphocyte levels present in blood samples taken from subjects in the study of the effects of bovine colostrum supplementation.

### DETAILED DESCRIPTION OF THE INVENTION

This invention establishes a correlation between the consumed dose of bovine colostrum and the resulting enhanced levels of immunoglobulins and lymphocytes present in the immune system of human subjects, and therefore demonstrates a method of enhancing the immune response in a human through the dose dependant consumption of bovine colostrum.

Experiment:

In this experiment forty (40) healthy female students from the Home Economics Faculty of Minufiya University were selected for the study. The females ranged in age between 17.5 and 19.4 years old. They were all non-smokers and did not have a history of any serious disease. The female subjects did not take any therapeutic drugs or other forms of food supplements for six months before the study or during the four weeks of the study. Each of the subjects was given full information about the research protocol before participation.

The forty female subjects were divided into two groups with twenty students per group. Group A received 200 mg of bovine colostrum daily. Group B received 400 mg of bovine colostrum daily. The bovine colostrum supplement was in the form of chewable lozenges supplied by Immuno-Dynamics, Inc., a corporation in Perry, Iowa. The lozenges were certified as free from all forms of pathogens, drugs, heavy metals, and pesticides.

The female subjects were directed to take their determined dosage of bovine colostrum supplement daily for two successive weeks. The duration of the study was four weeks which was divided into the following intervals: A) Pre-feeding, (time zero) the day the subjects started the colostrum supplement B) Feeding duration, the first and second week of colostrum supplementation, C) Post-feeding, the third and fourth week after taking the colostrum supplementation.

In order to test and record the effectiveness of the bovine colostrum supplementation, five blood samples (5 ml each) were taken from each subject, one at each of the above-identified intervals. For basal time the sample was taken immediately before receiving the first dose of bovine colostrum lozenges. The following four samples were taken at the end of the first and second weeks of the feeding duration and at the end of the third and fourth weeks of the post-feeding duration. The blood samples were taken from the antecubital vein of the right arm and transferred to a glass tube containing EDTA. The tube was immediately centrifuged at 5000 rpm for 10 minutes to collect the serum. The removed serum was stored in a plastic Eppendrof tube and frozen until the time of inspection.

The five serum samples collected from each subject were analyzed for immunoglobulins IgG, IgM, and IgA using the techniques and test kits provided by Endoplate Single Radial Immunodifussion (RID) (Nerenberg & Prasad 1975). The T lymphocytes, CD4 and CD8, were analyzed by using direct flow cytometric Immunoflourescence Specific Staining kit methods (Sabin et al, 1994 and Vigali & Strominger 1994). Finally, the obtained results were analyzed by pairing the students into a two-tailed T-test via Multivariate Analysis of Variance (MANOVA) to produce an F-statistic in order to detect the presence of a significant difference within the treatments. The level of significance was set at P<0.05 (Statograph Program 1987).

Results:

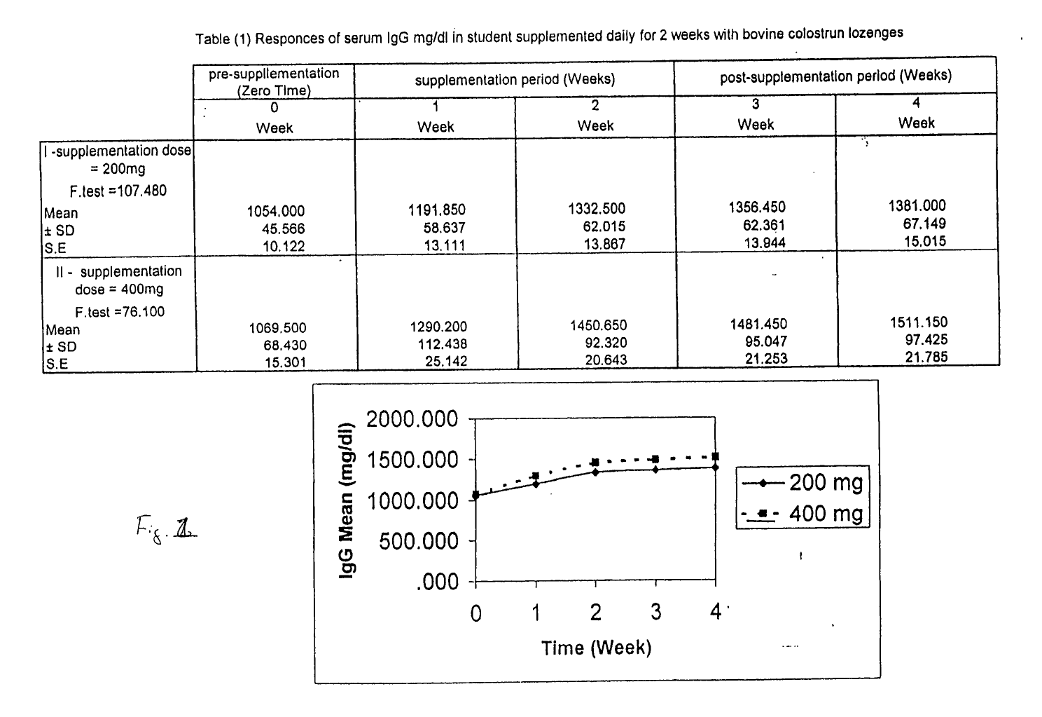

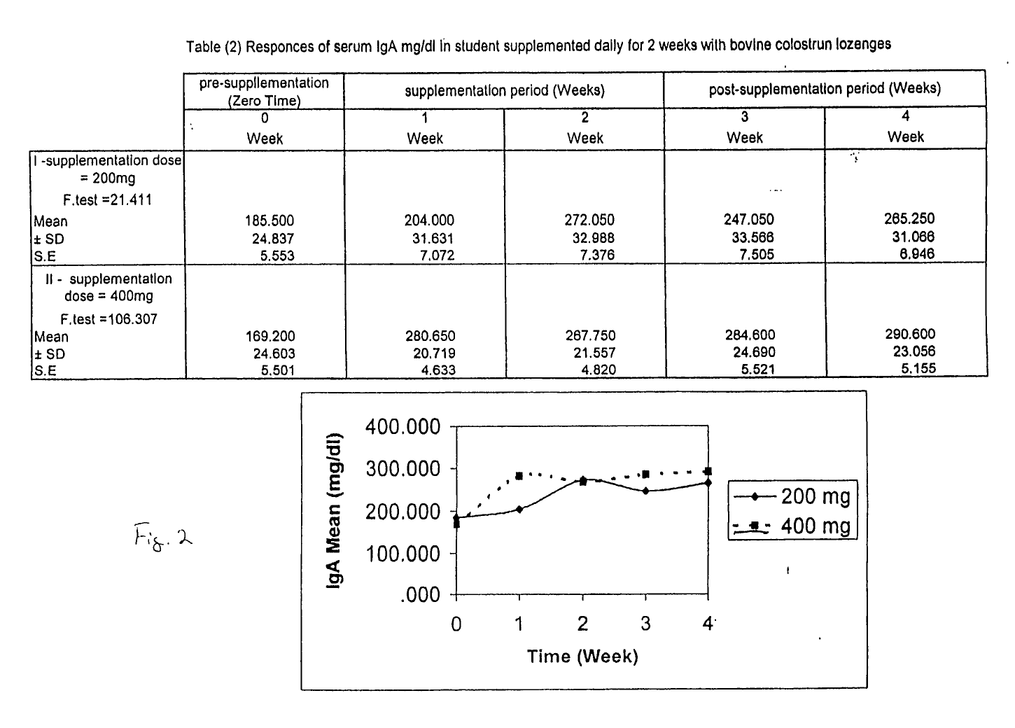

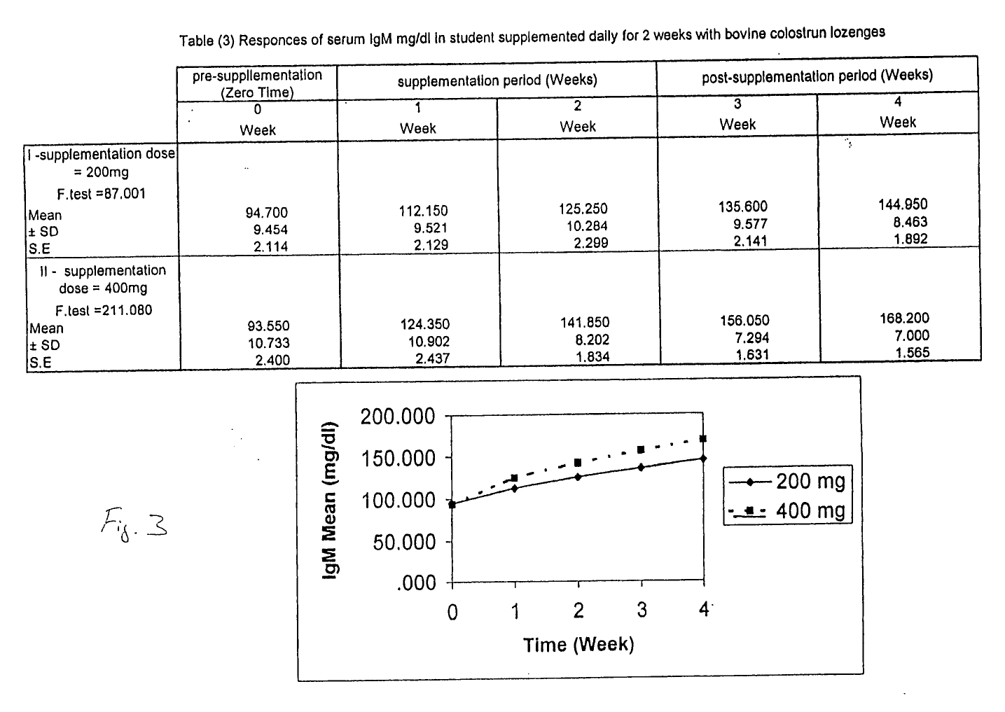

The data presented in Tables 1-3 and FIGS. 1-3 shows the analysis of serum immunoglobulin IgG, IgM, and IgA titers as affected by the daily intake of bovine colostrum lozenges for the two subject groups. A significant difference was observed in the estimated titer of the two subject groups and was determined to be dependent on the dose of bovine colostrum consumed. The analysis of the feeding duration shows that Group B (subjects who received 400 mg of bovine colostrum daily) had significantly higher levels (P<0.05) of the estimated immunoglobulins than the Group A (students that received a daily dose of 200 mg of bovine colostrum). During the post-supplementation period there was no significant difference (P<0.05) between the two subject groups in the detected increase of the estimated immunoglobulin classes.

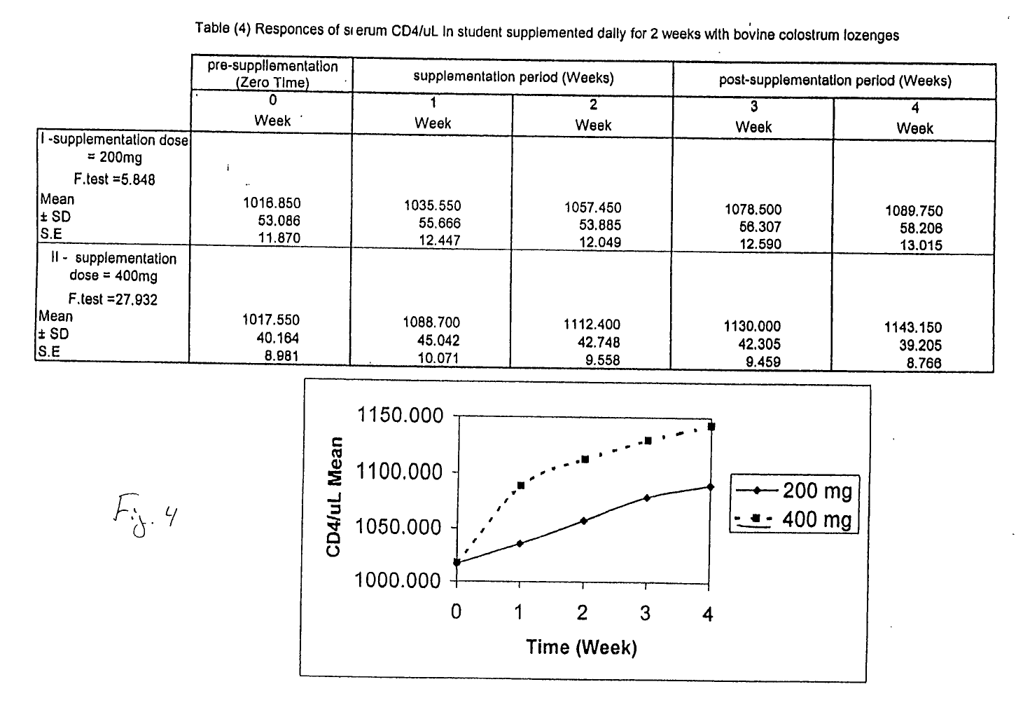

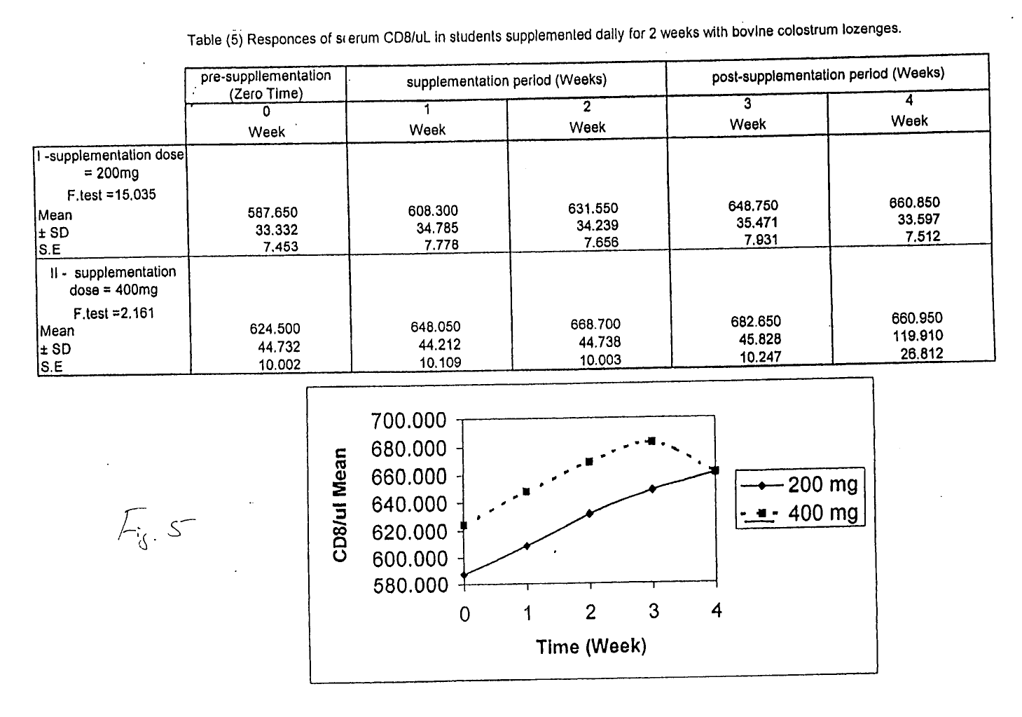

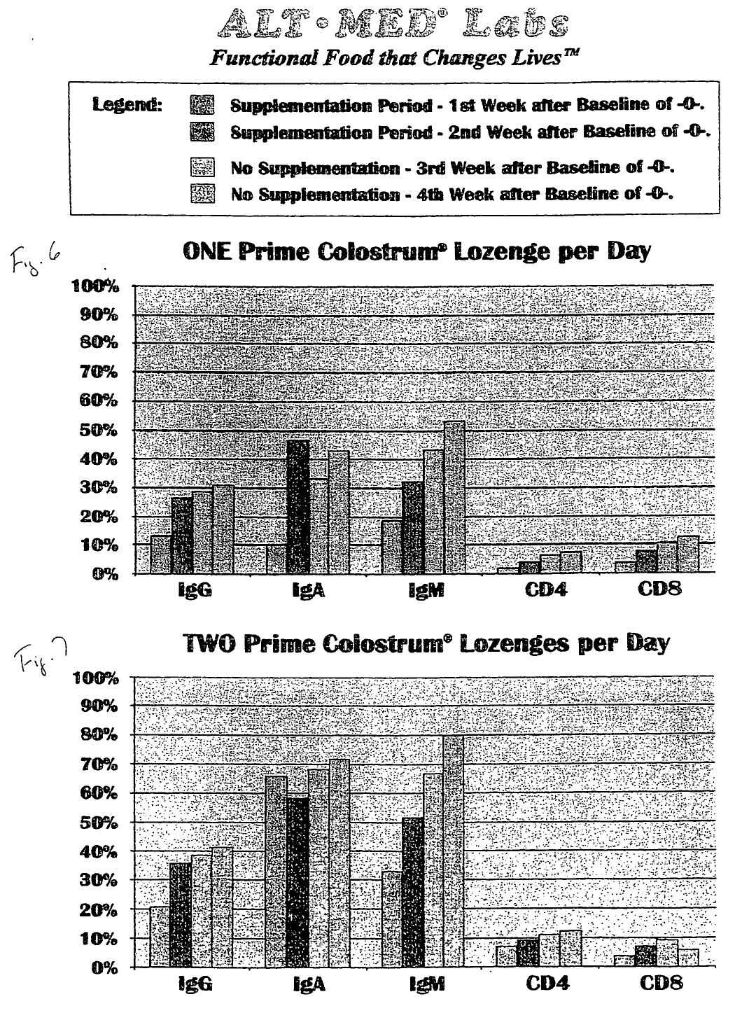

The data of the estimated lymphocyte groups (CD4 and CD8) is shown in Tables 4 and 5 and FIGS. 4 and 5. Again it was observed that Group B had a greater increase in the level of estimated lymphocytes than Group A. It was further noted that there was a continual increase in the observed level of the CD4 lymphocytes until the end of the fourth week of the study in both Groups A and B, whereas, the level of the CD8 lymphocytes underwent a gradual decline during the fourth week in both groups. The combined results of the lymphocytes and immunoglobulins present in the blood samples taken from the human subjects during the experiment are displayed in FIGS. 6 and 7. FIG. 6 shows the results of the blood samples taken from Group A and FIG. 7 shows the results of the blood samples taken from Group B.

Although prior to the study, it was believed that the oral ingestion of bovine colostrum could provide protection for humans against viral gastrointestinal infections due to the high contents of immunoglobulins naturally in abundance in the colostrum along with other natural antiviral constituents, the mechanism allowing for this enhanced immune response was not fully understood. Not wishing to be bound by any particular theory of operation, the results of the present invention are consistent with the possibility that most bovine colostrum immunoglobulins, and other biologically active constituents, have the ability to withstand the human gastric proteolytic enzymatic actions and therefore could be absorbed very effectively from the intestine into the blood stream in order to work as exogenous non-specific immunoglobulins, thus leading to a detectable increase of immunoglobulins in the serum level. Bovine colostrum is further identified as acting like a reactor to the immune system by both promoting the release of human defense host immunoglobulins, as well as providing immune protection through the stimulation of releasing the reactive immunoglobulins, which are present in the human immune defense.

There was a noted increase in the incidence of significance and improvement of all the examined immunity parameters based on the experiment's increased daily consumption of the bovine colostrum supplement. Although the estimated immunity parameters and indices were previously shown to be associated with various effective functions of the human immune system and general systemic body defense reactions, the observed increase in all investigated parameters of the present experiment, which was carried out in a dose dependant linear manner, demonstrated that a significant correlation was observed in the human immune response depending on the daily doses of bovine colostrum consumed (200 mg or 400 mg). The larger dose of bovine colostrum provided a higher immune system response.

The observed increase in serum immunoglobulins was directly related to bovine colostrum consumption and, as stated, is believed to be primarily due to the high content of immunoglobulins present naturally in bovine colostrum, which when ingested is absorbed by the body and enhances the immune rezones. This explanation of the means by which bovine colostrum supplementation enhances the immune response is supported by the fact that the IgG immunoglobulins were the most predominant immunoglobulin and was observed to significantly increase during the experiment. IgG immunoglobulins are known to have a higher resistance than the other classes of immunoglobulins to the acidic nature and gastric degradation of stomach enzymes. Another theory for explaining the significant increase in the titer of the serum immunoglobulins from the consumption of bovine colostrum is that some of the ingested bovine colostrum constituents are digested into biologically active fractions that act as immunogens so as to promote the individual's own immunity system to produce and release its own human immunoglobulins.

The individual's immune system may be activated by the transfer of Growth Factor-B (TGF-B), which is naturally abundant in bovine colostrum. TFG-B could serve in the activation and stimulation of B lymphocytes by producing immunoglobulins, specially IgG and IgA, in addition to the enhancement of transporting of polymeric IgA into the intestinal lumen in order to activate the immune response of both the intestine and other mucous membranes against pathogens and other environmental pollutants. This finding supports the suggestion that the role of active constituents present naturally in abundance in bovine colostrum may stimulate not only the B-lymphocytes to produce and release the immunoglobulins but may also initiate and activate the T-lymphocytes to produce and release CD4 and CD8 as members of T-lymphocytes which are involved mainly in the cell-mediated immune mechanism of humans.

The method of the present invention has expanded the clinical and nutritional role of bovine colostrum supplementation in humans by quantifying and demonstrating that the bovine colostrum supplementation serves the individual's health by effectively enhancing and providing modulation and improvement of the general immunity of humans. The improved immune response is likely achieved by increasing the immunoglobulins and lymphocytes present in the system as well as introducing high contents of anti-microbial factors and other complementary proteins. The correlation between the increase in the consumption of bovine colostrum and the increase in the immune response supports the method of the invention. Therefore, bovine colostrum products offer potential protection against infectious pathogens and protection associated with other benefits of the immune system.

The experimental study further demonstrates the nutritional benefits of bovine colostrum, and therefore places focus on the production of a wide range of bovine colostrum containing or based foods in order to provide support and enhancement for an individual's immune response, especially for infants and individuals who suffer from immune related diseases. The suggestion to incorporate the biologically active constituents of bovine colostrum into human foods also offers many physiological benefits.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

1. A method for enhanced immune response, comprising administering to a human subject at least 200 mg per day of bovine colostrum supplement for a period of two weeks until immune response in the subject is enhanced.

2. (canceled)

3. The method as defined in claim 1, wherein the enhancement in the immune response comprises an increase in the levels of immunoglobulins and lymphocytes present in a subject's blood serum.

4. The method as defined in claim 1, wherein the amount of colostrum supplementation is between 200 mg to 1000 mg per day.

5. The method as defined in claim 1, wherein the amount of colostrum supplementation is 400 mg per day.

6. (canceled)

7. (canceled)

8. (canceled)

9. (canceled)

10. (canceled)

11. The method as defined in claim 3, wherein the type of immunoglobulins with increased levels is IgG.

12. The method as defined in claim 3, wherein the type of immunoglobulins with increased levels is IgA.

13. The method as defined in claim 3, wherein the type of lymphocytes with increased levels is CD4.

14. The method as defined in claim 3, wherein the type of lymphocytes with increased levels is CD8.

15. The method as defined in claim 1, wherein the enhancement in the immune response comprises enhanced protection against infection.
